(19) United States
(12) Patent Application Publication
(10) Pub. No.: US 2016/0310630 A1
(43) Pub. Date: Oct. 27, 2016

(54) TABLET-SHAPE-COMPRESSED HEMOSTATIC MATERIAL FOR SURGICAL PURPOSE

(71) Applicant: Xinsheng ZHU, Guangzhou (CN)

(72) Inventor: Xinsheng ZHU, Guangzhou (CN)

(21) Appl. No.: 15/202,610

(22) Filed: Jul. 6, 2016

Related U.S. Application Data
(63) Continuation-in-part of application No. PCT/CN2014/093689, filed on Dec. 12, 2014.

(30) Foreign Application Priority Data
Jan. 6, 2014 (CN) 201410005991.2

(51) Int. Cl. A61L 15/44; A61B 17/00; A61L 15/42

(52) U.S. Cl. CPC A61L 15/44; A61L 15/425; A61B 17/0057; A61L 2400/04; A61L 2300/606; A61L 2300/418; A61B 2017/00654; A61B 2017/00884

(57) ABSTRACT

A hemostatic material, including (a) an absorbable hemostatic gauze and (b) an absorbable hemostatic sponge. The hemostatic material is provided in a tablet-shape.

### CROSS-REFERENCE TO RELATED APPLICATIONS

This application is a continuation-in-part of International Patent Application No. PCT/CN2014/093689 with an international filing date of Dec. 12, 2014, designating the United States, now pending, and further claims priority benefits to Chinese Patent Application No. 201410005991.2 filed Jan. 6, 2014. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, and Cambridge, MA 02142.

### BACKGROUND OF THE INVENTION

1. Field of the Invention

The invention relates to a medical material, and more particularly to a tablet-shape-compressed hemostatic material for surgical purpose.

2. Description of the Related Art

Existing hemostatic materials do not achieve hemostasis quickly and/or efficiently, and especially, are unsuitable for minimally invasive surgery.

### SUMMARY OF THE INVENTION

In view of the above-described problems, it is one objective of the invention to provide a tablet-shape-compressed hemostatic material for surgical purpose.

To achieve the above objective, in accordance with one embodiment of the invention, there is provided a tablet-shape-compressed hemostatic material for surgical purpose, comprising a tablet-shape-compressed hard hemostatic gauze, and the hard hemostatic gauze is formed by compressing an absorbable hemostatic gauze and an absorbable hemostatic sponge.

Preferably, an end surface of the hemostatic sponge back against the hemostatic gauge is provided with a hard hemostatic sponge layer.

Preferably, the hemostatic gauge is coated with a coagulant.

Preferably, the hemostatic gauge is made from compressible hard material.

Preferably, the hemostatic sponge is made from compressible sponge material.

Preferably, the hard hemostatic sponge layer is integrated with the hard hemostatic gauze.

Preferably, the hard hemostatic sponge layer is separated from the hard hemostatic gauze.

Advantages of the tablet-shape-compressed hemostatic material for surgical purpose according to embodiments of the invention are summarized as follows:

When the hard hemostatic gauze formed by compressing the absorbable hemostatic gauze and the absorbable hemostatic sponge presses the bleeding wound, the hemostatic sponge is adapted to quickly absorb water and swell, enabling the hemostatic gauze to attach to the bleeding wound. The hemostatic sponge quickly absorbs moisture in blood, thus hemocyte, clotting factors and the like are quickly concentrated, and a coagulation mechanism is started, as a result, stemming of blood flow is achieved; at the same time, the coagulant on the hemostatic gauze produces gel on the wound and on a bleeding spot, and the gel adheres to and blocks the bleeding spot, thereby the hemostasis effect is improved due to a common action of the hemostatic gauze and the hemostatic sponge.

### BRIEF DESCRIPTION OF THE DRAWINGS

The invention is described hereinbelow with reference to the accompanying drawings, in which:

FIG. 1 is a side view of a tablet-shape-compressed hemostatic material for surgical purpose in accordance with one embodiment of the invention; and

FIG. 2 is a schematic diagram of a tablet-shape-compressed hemostatic material for surgical purpose with a hard hemostatic sponge layer in accordance with one embodiment of the invention.

### DETAILED DESCRIPTION OF THE EMBODIMENTS

For further illustrating the invention, experiments detailing a tablet-shape-compressed hemostatic material for surgical purpose are described below. It should be noted that the following examples are intended to describe and not to limit the invention.

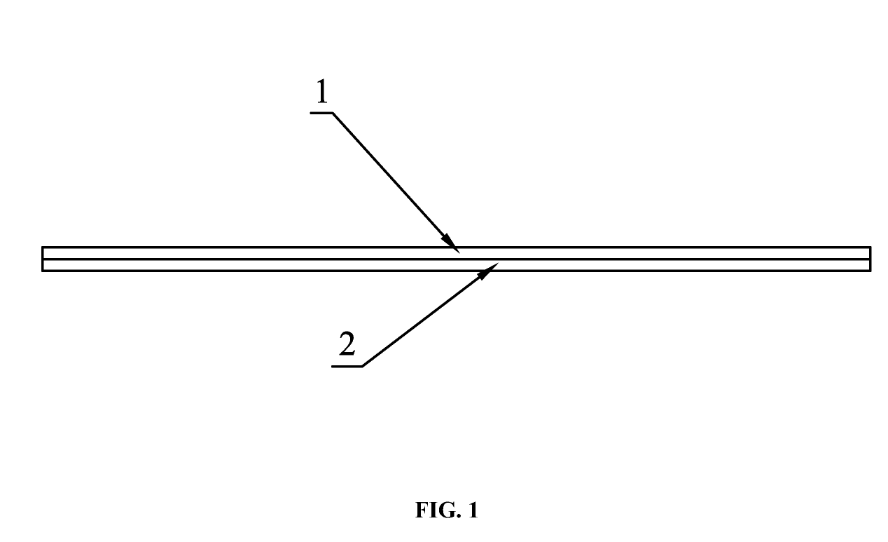

As shown in FIG. 1, a tablet-shape-compressed hemostatic material for surgical purpose, comprising a tablet-shape-compressed hard hemostatic gauze, and the hard hemostatic gauze is formed by compressing an absorbable hemostatic gauze 1 and an absorbable hemostatic sponge 2.

When the hard hemostatic gauze formed by compressing the absorbable hemostatic gauze 1 and the absorbable hemostatic sponge 2 presses the bleeding wound, the hemostatic sponge 2 is adapted to quickly absorb water and swell, enabling the hemostatic gauze 1 to attach to the bleeding wound. The hemostatic sponge 2 quickly absorbs moisture in blood, thus hemocyte, clotting factors and the like are quickly concentrated, and a coagulation mechanism is started, as a result, stemming of blood flow is achieved; at the same time, the coagulant on the hemostatic gauze 1 produces gel on the wound and on a bleeding spot, and the gel adheres to and blocks the bleeding spot, thereby the hemostasis effect is improved due to a common action of the hemostatic gauze 1 and the hemostatic sponge 2.

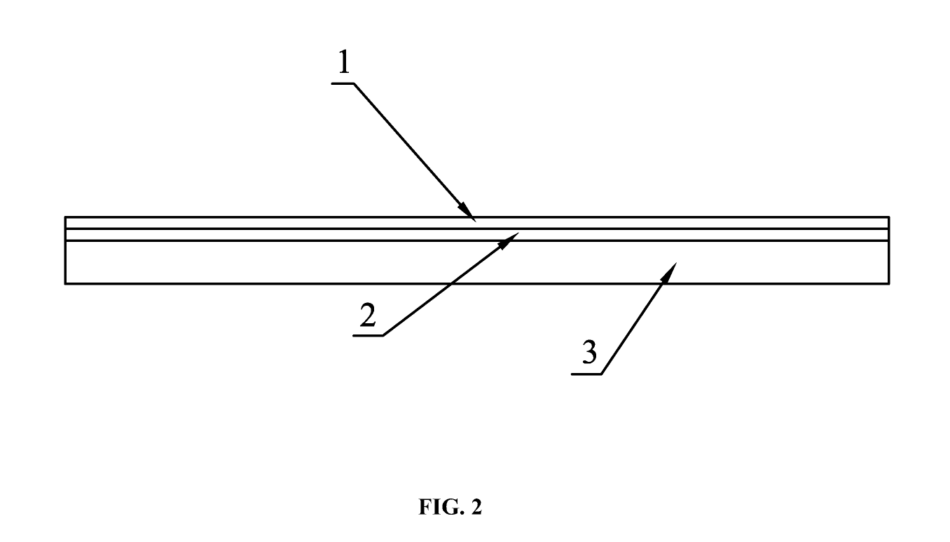

Preferably, as shown in FIG. 2, an end surface of the hemostatic sponge 2 back against the hemostatic gauge 1 is provided with a hard hemostatic sponge layer 3.

An end surface of the hemostatic sponge 2 is provided with a hard hemostatic sponge layer 3, and when the hard hemostatic sponge layer 3 presses the bleeding wound, the hard hemostatic sponge layer 3 is adapted to quickly absorb water and swell, enabling the hemostatic gauze 1 to attach to the bleeding wound. The hard hemostatic sponge layer 3 and the hemostatic sponge 2 work together to stem the blood flow, and the coagulant on the hemostatic gauze 1 produces gel and blocks the bleeding spot, as a result, the hemostasis effect is improved due to a common action of the hemostatic gauze 1, the hemostatic sponge 2, and the hard hemostatic sponge layer 3.

Preferably, the hemostatic gauge 1 is coated with a coagulant.

Preferably, the hemostatic gauge 1 is made from a compressible hard material.

Preferably, the hemostatic sponge 2 is made from a compressible sponge material.

Preferably, the hard hemostatic sponge layer 3 is integrated with the hard hemostatic gauze. Before the hard hemostatic sponge layer 3 starts to work and absorb water, the hard hemostatic sponge layer 3 is integrated with the hard hemostatic gauze.

Preferably, the hard hemostatic sponge layer 3 is separated from the hard hemostatic gauze. When the hard hemostatic sponge layer 3 has absorbed enough water, the hard hemostatic sponge layer 3 is separated from the hard hemostatic gauze.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A hemostatic material, comprising (a) an absorbable hemostatic gauze and (b) an absorbable hemostatic sponge, wherein the hemostatic material is compressed in a tablet-shape.

2. The hemostatic material of claim 1, wherein an end surface of the hemostatic sponge back against the hemostatic gauge is provided with a hard hemostatic sponge layer.

3. The hemostatic material of claim 1, wherein the hemostatic gauge is coated with a coagulant.

4. The hemostatic material of claim 2, wherein the hemostatic gauge is coated with a coagulant.

5. The hemostatic material of claim 1, wherein the hemostatic gauge is made from compressible hard material.

6. The hemostatic material of claim 2, wherein the hemostatic gauge is made from compressible hard material.

7. The hemostatic material of claim 1, wherein the hemostatic sponge is made from compressible sponge material.

8. The hemostatic material of claim 2, wherein the hemostatic sponge is made from compressible sponge material.

9. The hemostatic material of claim 1, wherein the hard hemostatic sponge layer is integrated with the hard hemostatic gauze.

10. The hemostatic material of claim 2, wherein the hard hemostatic sponge layer is integrated with the hard hemostatic gauze.

11. The hemostatic material of claim 1, wherein the hard hemostatic sponge layer is separated from the hard hemostatic gauze.

12. The hemostatic material of claim 2, wherein the hard hemostatic sponge layer is separated from the hard hemostatic gauze.
